(19) United States
(12) Patent Application Publication
(10) Pub. No.: US 2016/0302484 A1
(43) Pub. Date: Oct. 20, 2016

(54) AIRFLOW TESTING APPARATUS AND METHOD FOR AN INHALER

(71) Applicant: KIND CONSUMER LIMITED, London (GB)

(72) Inventors: Ritika GUPTA, London (GB); Rene Mauricio GONZALEZ CAMPOS, London (GB); Alex HEARN, London (GB); David HACKETT, Kent (GB)

(21) Appl. No.: 15/103,034

(22) PCT Filed: Nov. 25, 2014
(86) PCT No.: PCT/GB2014/053481

§ 371 Date: Jun. 9, 2016

(30) Foreign Application Priority Data
Dec. 10, 2013 (GB) 1321824.3

(51) Int. Cl. A24F 47/00; G01N 15/02; A61M 15/06

(52) U.S. Cl. CPC A24F 47/002; A61M 15/06; G01N 15/0255; G01N 15/0205; A61M 2209/02; G01N 2015/0261

(57) ABSTRACT

Testing apparatus comprising an air pump downstream of measuring equipment which measures the characteristics of an inhaler is provided. An induction port upstream of the measuring equipment has an adaptor connected at a first end. The adaptor comprises a through-bore extending through the adaptor from the first end to a second end to receive an inhaler and a bypass channel in communication with the through-bore. The invention also includes a testing method and the adapter.

The present invention relates to testing apparatus for air flow.

In particular, it has been designed as test equipment for an inhaler, more specifically, a simulated cigarette device such as a nicotine inhaler or an electronic cigarette.

Currently most pharmaceutical inhaled products rely on user co-ordination for actuation and inhalation. The user must trigger the device, for example, by depressing a canister of a product and breathing in the dispensed product. In order to test such a dispenser for its performance, it is held at the inlet of a suitable piece of test apparatus containing measuring equipment such as an Andersen Cascade Impactor, Next Generation Impactor or Spraytec Malvern. The measuring equipment can either directly measure or enable sampling for measurement. An independent air flow can be set through the measuring equipment within the testing apparatus using a pump and the device is actuated in order to dispense the product into the air flow.

The air pump generates a relatively high flow rate as required by the measuring equipment. This is not a problem for a conventional inhaler and this air flow can simply pass through the inhaler without affecting the dispensing ability or damaging the inhaler.

However, such testing apparatuses are not suitable for all types of inhalers. In particular, they are not suitable for inhalers which have breath-activated valves or triggering mechanisms that may be mechanical, chemical or electronic, particularly those which are triggered at a low flow rate. Such inhalers are, for example, a simulated cigarette developed by the applicant as disclosed, for example, in WO 2011/015825. This has a breath-activated valve which has been specifically designed in order to trigger at a low flow rate which coincides with the flow rate for a conventional cigarette such that the device is as close as possible to the smoking experience. Electronic cigarettes, of which there are numerous variations, provide another such example.

Currently, no means of testing the emissions from such devices is available. The above mentioned testing apparatuses are unsuitable because the high flow rate required is incompatible with the excessively high resistance to flow through the device. Therefore the device can become damaged if it is subjected to the air flow rates required by the measuring equipment. Additionally, the apparatuses cannot produce useful data at the low flow rates the device requires.

We are currently aware of only one attempt to provide an air flow testing apparatus which allows a high resistance device to be tested by an apparatus requiring high air flow rates. This is a product called the Mixing Inlet produced by Copley Scientific (European Journal of Pharmaceutical Sciences-39 (2010) 348-354).

This has been designed for traditional dry powder inhalers which typically actuate at 60 litres per minute as opposed to a simulated cigarette device which would typically actuate at around 2 litres per minute. In the case of testing using an Anderson Cascade Impactor (ACI), for example, the approach taken is to place the Mixing Inlet within the testing apparatus itself. It is fitted between the induction port and the remaining ACI stack. The Mixing Inlet has a central duct for the composition flow which is surrounded by a generally conical chamber having a supplementary air inlet. The supplementary air inlet is connected to a compressed air source and injects compressed air around the product stream. An air pump, attached downstream of the ACI stack, is set at the flow rate required for functioning of the ACI setup. Upon activation of this pump, air begins to flow through the setup upstream of it i.e. through the device, the induction port section, and the ACI stack. Since the compressed air is provided through the supplementary inlet of the equipment, only a residual volume of air is forced through the device and central core of the Mixing Inlet. It is at this point that air through the secondary inlet and formulation meet to enter the ACI stack at a required flow rate.

The separate source of compressed air adds expense and complexity to the equipment. Further, as it is positioned downstream of the induction port, the induction port sees only the low flow rate from the inhaler. Because of this low flow rate, an unrepresentative amount of the product from the inhaler may be deposited in the induction port, thereby distorting the subsequent deposition profile.

According to a first aspect of the present invention, there is provided testing apparatus comprising an air pump downstream of measuring equipment which measures a product's characteristics; an induction port upstream of the measuring equipment; and an adaptor connected at a first end upstream of the induction port, the adaptor comprising a through-bore extending from the first end to a second end to receive an inhaler, in use, and a bypass line in communication with the through-bore, whereby the air pump, in use, draws air through the inhaler and through the bypass line.

The present invention uses the adaptor in place of the above mentioned mixing inlet. This provides two key advantages. Firstly, because it relies on a bypass flow, it uses only the air flow generated by the air pump of the testing apparatus and therefore eliminates the need for a compressed air source and its associated couplings and control. Secondly, as it is an adaptor which is couplable to an inlet of the testing apparatus, it sits upstream of the induction port so that the make-up air flows through the induction port. The induction port therefore is exposed to drug formulation in a stream of air at the flow rate required by the testing apparatus such that the rate of deposition of the product within the induction port is within normal design parameters.

If the dimensions of the inhaler and its flow characteristics are well known, then the adaptor may be designed with fixed port sizes which are suited to that particular inhaler. In order to set a flow characteristic of a different type of adaptor, a second adaptor with different flow characteristics may be used in place of the first one. However, preferably, the adaptor is provided with a flow adjustment member in the bypass line, the member being adjustable to vary the flow through the bypass line, and hence the relative proportions of air that are drawn in at the first end of the adaptor and the make-up air drawn in through the bypass line.

The flow adjustment member may, for example, be a replaceable component, a number of which are available in different sizes. Thus, the user can select an appropriately sized component to block enough of the bypass line to provide the required flow characteristics. However, preferably, the flow adjustment member is a member which is movable with respect to the through-bore such that it can be adjusted, in situ. Preferably, it is a screw threaded nut as this provides a fine degree of control of the flow path.

The bypass line may open directly into the through-bore. However, preferably, the bypass line includes an annular chamber having an outlet surrounding the through-bore. This ensures that the flow through the bypass line is evenly distributed around the inhaler's plume, thereby avoiding undue deflection of the plume.

Preferably, the adaptor has a support arm extending from the first end to support an inhaler, in use.

According to the second aspect of the present invention, there is provided:

a method of testing an inhaler using testing apparatus having an inlet upstream of an induction port which, in turn, leads into measuring equipment, with an air pump downstream of the measuring equipment to draw air in through the inlet, along the angled induction port and into the measuring equipment;

the method comprising fixing a first end of an adaptor to the inlet, the adaptor comprising a through-bore extending through the adaptor from the first end to a second end which receives the inhaler, a bypass line in communication with the through-bore;

attaching an inhaler with its outlet in fluid communication with the second end of the adaptor; and

drawing air through the inhaler and bypass line, into the induction port and subsequently into the testing equipment.

Preferably the method comprises drawing up to 100 litres/minute and preferably up to 70 litres/minute through the inhaler and bypass line and into the testing apparatus.

Preferably, the method comprises drawing at least 80%, and preferably at least 90% of the flow through the bypass line and the remainder through the inhaler.

The bypass line of the adaptor may have the fixed geometry or the flow adjustment member as set out above in relation to the first aspect of the invention.

According to a third aspect of the present invention, there is provided an adaptor for the inlet of air flow testing equipment, the adaptor comprising a sleeve with a through-bore extending through the adaptor from a first end couplable to an inlet of the testing equipment, to a second end having a seal to receive and seal, in use, with an inhaler, a bypass line in communication with the through-bore and a flow adjustment member in the bypass line, the member being adjustable to vary the flow through the bypass line, and hence the relative proportion of air that is drawn in at the second end of the adaptor and the make-up air drawn in through the bypass line.

The flow adjustment member and other details of the adaptor are preferably in accordance with the preferred features set out above in relation to the first aspect of the invention.

Examples of the adaptor, equipment and method in accordance with the present invention will now be described with reference to the accompanying drawings, in which:

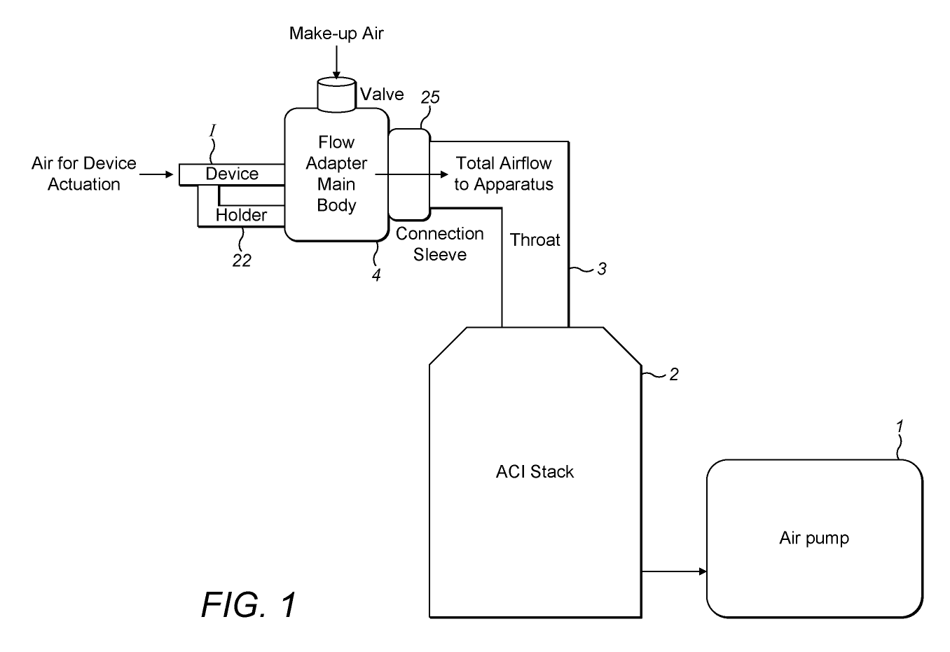

FIG. 1 is a schematic representation of the equipment; and

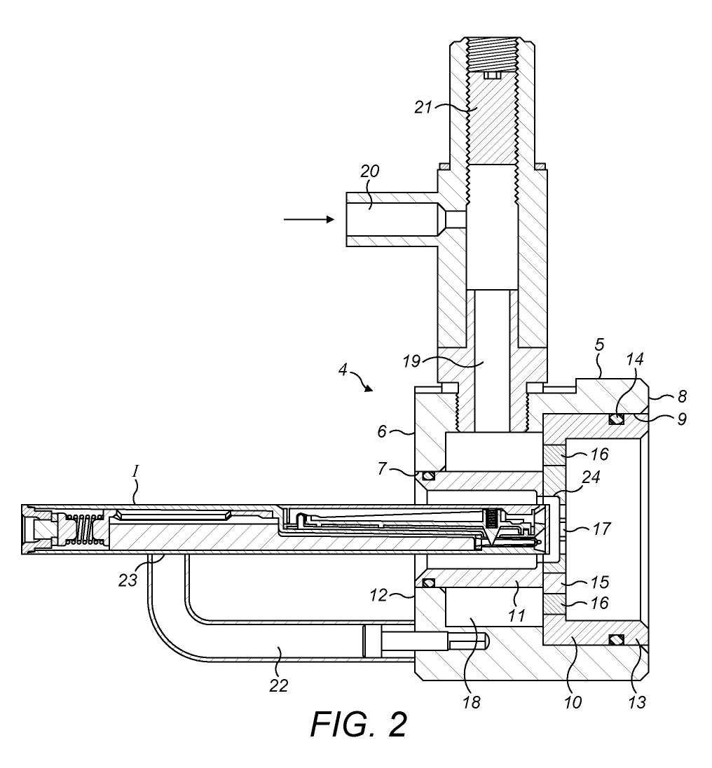

FIG. 2 is a cross-section through the adaptor.

FIG. 1 is a schematic representation of the testing equipment, most of which is standard testing equipment. This consists of three main components, namely an air pump 1, testing equipment 2 such as a particle laser diffraction volume or size determinations equipment. More specifically, it may be an Andersen Cascade Impactor (ACI), a Spraytec Malvern or a next generation Impactor. It could be used with any test that requires a DUSA (Dose Uniformity Sampling Apparatus).

The present example shows an ACI. The induction port 3 is in the form of a duct which has a right-angled bend and leads into the ACI stack. Thus, when the air pump is operated, air is drawn through the induction port and into the stack. In the ACI, the aerosol is sampled at different stages, tests are carried out on the sample collected on each stage with different analytical equipment. Flow characteristics measured include total dose recovered, fine particle dose (drug content in particles below a specific size), drug content in specific particles size brackets, Mass Median Aerodynamic Diameter (MMAD) in a manner well known in the art.

The non-conventional part relates to the adaptor 4 as shown in FIG. 2.

The adaptor 4 comprises a main body 5 having a generally hollow cylindrical configuration with a first end 6 having a narrow central opening 7 and a second end 8 with a wider opening 9. An insert 10 is inserted through the wide opening 9. This has a narrow cylindrical duct 11 extending towards the first end and which is provided with an O-ring seal 12 to seal with the narrow opening 7. The opposite end has a wider cylindrical body 13 and has an O-ring seal 14 to seal in the wider opening 9. The passage through the cylindrical duct 11 and cylindrical body 13 forms a through bore to receive an inhaler I as described below. Between the duct 11 and body 13, is an annular plate 15 which has a plurality of orifices 16 (in this case 6 such orifices) arranged around its periphery and a single central opening 17.

Between the narrow cylindrical duct 11 and the inner wall of the main body 5 is an annular chamber 18 which communicates with a bypass channel 19. The bypass channel 19 is a straight bore from which a lateral bore 20 extends. A screw threaded nut 21 is positioned in the straight bore 19 and can be screwed down this bore to selectively block part of the exit of the lateral bore 20. In this way, the size of the narrowest part of the bypass line 19 can be controlled and hence the relative proportion of air drawn in through this bore. A similar effect could be achieved by providing the nut in the lateral bore and selectively advancing it into the straight bore.

An arm 22 extends from the bottom portion of the first end 6 of the adaptor and extends upwardly to terminate in a support surface 23 for supporting an inhaler I in the adaptor. As can be seen in FIG. 2, the inhaler is positioned with its outlet end adjacent to the central opening 17 and is sealed with respect to the adaptor 4 by a ring 24.

The second end 8 of the adaptor may be provided with some feature, such as a screw thread, for connection to the equipment. In this example, a silicon sleeve 25 fits both the equipment and the adaptor to secure the adaptor in place (sleeve not shown in FIG. 2).

Various different sizes of inhaler can be accommodated in the adaptor, if necessary, simply by changing the arm 22 and seal 24 and potentially also the insert 10 to accommodate inhalers having different dimensions. Further variations can be accommodated by also replacing the insert 10 to accommodate an even larger inhaler.

With the adaptor 4 and inhaler I in place on the equipment, the nut 21 can be adjusted to vary the critical size of the lateral bore. In carrying out the testing, the air pump 1 is operated in order to satisfy the flow requirements required of the ACI stack 2 and induction port 3, while the bypass channel 19 ensures that the flow through the inhaler I is kept to a level that will not damage it.

### EXAMPLE 1

For an Andersen-Cascade Impactor, the current European Pharmacopeia states in 2.9.18: The aerodynamic cut-off diameters of the individual stages of this apparatus are currently not well-established at flow rates other than 28.3 L/min. Users must justify and validate the use of the impactor in the chosen conditions, when flow rates different from 28.3 L/min are selected.

In practice, validating a new flow rate for a standard apparatus would require considerable time and expense. With the flow adaptor described, it is possible to test fine particle dose in an Anderson Cascade Impactor following the requirements around flow presented to the apparatus. The flow adaptor can be set to run 4 litres per minute through the device and 24.3 litres per minute through the flow-adaptor's diversion, ensuring the method equipment has all the airflow required for an appropriate measurement. Furthermore, an adaptor in any embodiment that includes flow or pressure sensors can provide further empirical evidence that the apparatus is presented with an appropriate flow rate.

### EXAMPLE 2

A Spraytec Malvern can be used to measure particle size in a sample aerosol presented. The System works on laser diffraction technology. However, experience demonstrates that it is important to achieve a stable aerosol, and this can be difficult without an air flow rate below 15 L/min. In this case, the flow adaptor can be set to run 4 litres per minute through the device and at 11 litres per minute through the flow-adaptor's diversion.

1. Testing apparatus comprising an air pump downstream of measuring equipment which measures a product's characteristics:
an induction port upstream of the measuring equipment; and
an adaptor connected at a first end upstream of the induction port, the adaptor comprising a through-bore extending through the adaptor from the first end to a second end to receive an inhaler, in use, and a bypass channel in communication with the through-bore, whereby the air pump, in use, draws air through the inhaler and through the bypass channel.

2. Apparatus according to claim 1, wherein the adaptor is provided with a flow adjustment member in the bypass channel, the member being adjustable to vary the flow through the bypass channel, and hence the relative proportions of air that are drawn in at the first end of the adaptor and the make-up air drawn in through the bypass channel.

3. Apparatus according to the claim 2, wherein the flow adjustment member is a member which is movable with respect to the bypass channel such that it can be adjusted, in situ.

4. Apparatus according to claim 1, wherein the bypass channel has an annular chamber bypass channel having an outlet surrounding the through-bore.

5. Apparatus according to claim 1, wherein the adaptor has a support arm extending from the first end to support an inhaler in use.

6. Apparatus according to claim 1, wherein the testing equipment is one of an Anderson-Cascade Impactor, Next Generation Impactor, Particle Size Determination by laser diffraction or Dose Uniformity Sampling Apparatus.

7. A method of testing an inhaler using testing apparatus having an inlet upstream of an induction port which, in turn, leads into measuring equipment, with an air pump downstream of the measuring equipment to draw air in through the inlet, along the induction port and into the measuring equipment:
the method comprising fixing a first end of an adaptor to the inlet;
the adaptor comprising a through-bore extending through the adaptor from the first end to a second end which receives the inhaler;
a bypass channel in communication with the through-bore;
attaching an inhaler with its outlet in fluid communication with the second end of the adaptor; and
drawing air through the inhaler and bypass channel, into the induction port and subsequently into the testing equipment.

8. A method according to claim 7, comprising drawing up to 100 litres/minute and preferably up to 70 litres/minute through the inhaler and bypass channel and into the testing apparatus.

9. A method according to claim 7, comprising drawing up to 80% and preferably up to 90% of the flow through the bypass channel and the remainder through the inhaler.

10. An adaptor for the inlet of air flow testing equipment, the adaptor comprising:
a sleeve with a through-bore extending through the adaptor from a first end couplable to an inlet of the testing equipment, to a second end having a seal to receive and seal, in use, with an inhaler
a bypass channel in communication with the through-bore and a flow adjustment member in the bypass channel, the member being adjustable to vary the flow through the bypass channel, and hence the relative proportion of air that is drawn in at the first end of the adaptor; and
the make-up air drawn in through the bypass channel.
